(19) United States
(12) Patent Application Publication
(10) Pub. No.: US 2004/0161075 A1
(43) Pub. Date: Aug. 19, 2004

(54) Radiographing apparatus

(75) Inventor: Kouji Amitani, Tokyo (JP)

Correspondence Address: FRISHAUF, HOLTZ, GOODMAN & CHICK, PC, 767 THIRD AVENUE, 25TH FLOOR, NEW YORK, NY, 10017-2023, US

(73) Assignee: KONICA MINOLTA HOLDINGS, INC., Tokyo (JP)

(21) Appl. No.: 10/776,410

(22) Filed: Feb. 10, 2004

(30) Foreign Application Priority Data
Feb. 18, 2003 (JP) JP2003-039808

(51) Int. Cl. A61B 6/04

(52) U.S. Cl. 378/37

(57) ABSTRACT

A radiographing apparatus for mammography is provided with a radiographing main body including a radiation source to emit radiation; a breast table on which a beast to be radiographed is placed, the breast table located opposite to the radiation source, and a first detector supporting rack to support a detector to receive radiation having passed through the breast when the beast is radiographed as an absorption contrast image, a supporting stand to support the radiographing main body at an upper position so as to provide a space at a lower position; and a detector supporting member shiftably provided in the space at the lower position of the supporting stand and having a second detector supporting rack to support a detector when the beast is radiographed as a phase contrast image.

### BACKGROUND OF THE INVENTION

This invention relates to a radiographing apparatus, especially relates to a radiographing apparatus, which is capable of phase contrast radiography.

A radiographing apparatus, which generally uses the action of radioactive rays to pass through a substance, is widely used for medical image diagnoses, non-destructive tests, etc. Especially, in regard to a radiographing apparatus for mammography used for radiography of a specific region of a human body, there has been a method wherein a photographic object is usually hold on a photographic object table united with a radiation image detection member to be radiographed. However, according to this method, there has been a problem that the sharpness of the image was inadequate as a radiographing apparatus for medical treatments used to read out the detailed structure of a specific region of a human body since the photographic contrast may not be good enough although a photographic object was radiographed on a life-sized basis.

It has been known a method wherein a radiation tube used in ordinary medical facilities (a small focal sized radiation source whose focal point size is 30-300 μm) was used in order to obtain phase contrast radiophotography. (Refer to patent reference No. 1) According to this, compared with the image of only usual absorption contrast, the contrast of photographic object's boundary can be described highly and it becomes possible to obtain a high definition radiation image vividly. However, when obtaining a phase contrast image, it is necessary to establish a fixed distance between a photographic object and a radiation image information detection member. Moreover, it is desirable that a radiography apparatus, which has not only a “phase image radiograpy mode” that photos a phase contrast image but also an “absorption contrast mode” that generally photos absorption contrast image when taking account of mitigation of examinee's burden and of cost for medical facilities.

Therefore, for example, in prior art, by providing a radiation image information detection member for an absorption contrast image attached detachably right under or right over the photographic object table to hold a photographic object and also by providing a detection member support table on which radiation image information detection member for phase change image information was supported detachably, under the photographic object table, with keeping a fixed distance between a radiation image information detection member for phase contrast image and a photographic object. Thereby, the same radiographing apparatus was arranged to have two modes, a phase image radiographing mode and a normal radiographing mode, by switching a setting location of the radiation image information detection member between the detection member support table and the vicinity to the photographic object table.

Patent Reference No 1.

Japanese Published patent application 2001-238871

However, in the case of normal radiographing mode, an examinee tends to hit his or her body against a detection member support table since the detection member support table is located right under the photographic object table even though the radiation image information detection member is not necessary. Especially when an examinee is radiographed sitting on a chair or a wheelchair, a detection member support table is going to be an obstacle to the examinee and sometimes the examinee has to stand up to be radiographed, which is a heavy burden to the examinee.

### SUMMARY OF THE INVENTION

It is an objective of this invention to provide a radiographing apparatus having the above drawback obviated and also having a detection member support table set at a location where the existence of the detection member support table to hold the radiation image information detection member for phase contrast radiographing which does not become an obstacle to the examinee and does decrease the examinee's burden when using a normal radiographing mode.

In order to solve the problem above and to achieve the objectives of this invention, this invention is comprised as follows.

A radiographing apparatus according to Item 1 comprising:

a photographic object table supporting a photographic object so that it faces a radiation source;

a radiation image information detection member detecting a radioactive ray passing through the photographic object;

an absorption contrast image support table supporting the radiation image information detection member so that it is located to be near the photographic object table and on opposite side of the radiation source for the photographic object when radiographing an absorption image;

a phase contrast image support table supporting the radiation image information detection member with a certain distance from the photographic object table so that at least a part of the radiation image information detection member is arranged to be inside irradiated area of the radiation source when taking a phase contrast image radiograph; and

a supporting member equipped with the phase contrast image support table can be mounted on and dismounted from a main body of mammography unit freely.

According to the invention of Item 1, the supporting member equipped with a phase contrast image support table can be mounted on and dismounted from a mammography unit freely so that a phase contrast image support table cannot be an obstacle and it gives less burdens to an examinee, when radiographing an absorption contrast image, namely, when the phase contrast image support table is not used.

A radiographing apparatus according to Item 2 is characterized in that the weight of the supporting member is 30 Kg or less in the radiographing apparatus according to Item 1.

According to the invention of Item 2, the weight of supporting member is as light as 30 Kg or less, which reduces burden for mounting and dismounting of the supporting member, and assure safe mounting and dismounting operation.

A radiographing apparatus according to Item 3 is characterized in that a grip handle is equipped on the supporting member in the radiographing apparatus according to Item 1 or Item 2.

According to the invention of Item 3, a grip handle is attached on the supporting member so that the supporting member can be attached or detached easily and safely, giving less burden to an operator of the apparatus.

The radiographing apparatus according to Item 4 is characterized in that the supporting member and the mammography unit are connected electrically in on of the radiographing apparatus according to either Item 1 or Item 3.

According to the invention of Item 4, the supporting member and the mammography unit are connected electrically so that a status of the supporting member attached or detached can be detected at the mammography unit and the malfunction can be prevented in advance if a sensor to detect the supporting member attached or detached, is provided. Furthermore, the phase contrast image support table attached to the supporting member can be operated from the mammography unit through remote control operations, and it is possible to move the phase contrast image support table to a certain position, or be made to retreat the phase contrast image support table when it is not used.

The radiographing apparatus according to Item 5 is characterized in that the phase contrast image support table attached to the supporting member can be retreated from the phase contrast image radiographing position to secure a space at the opposite side of the radiation source on the photographic object table.

The radiographing apparatus according to Item 5, when an absorption contrast image is photographed, in other words, when the phase contrast image support table is not used, it is possible to prevent that the phase contrast image support table becomes an obstacle to the examinee, and reduce a load factor to attach or detach the phase contrast image support table if the phase contrast image support table is retreated, even if the supporting member is not removed.

### BRIEF DESCRIPTION OF THE DRAWINGS

FIG. 1 is a Side view of a radiographing apparatus.

FIG. 2 is a Side view of a mammography unit.

FIG. 3(a) and FIG. 3(b) each is a perspective view of a supporting member.

FIG. 4 is a perspective view of another embodiment of a supporting member.

FIG. 5 is a side view of a radiation image photographic apparatus showing an electric connection of a supporting member.

### DETAILED DESCRIPTION OF THE PREFERRED EMBODIMENT

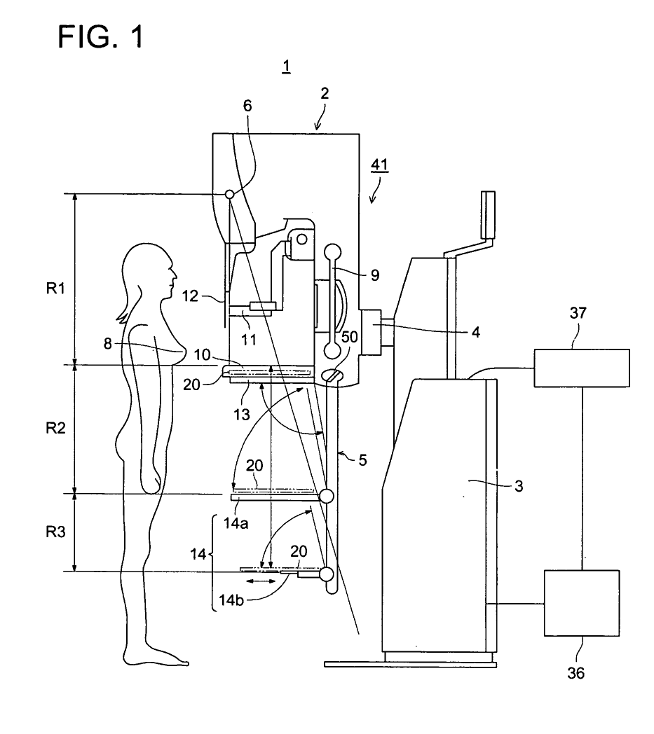

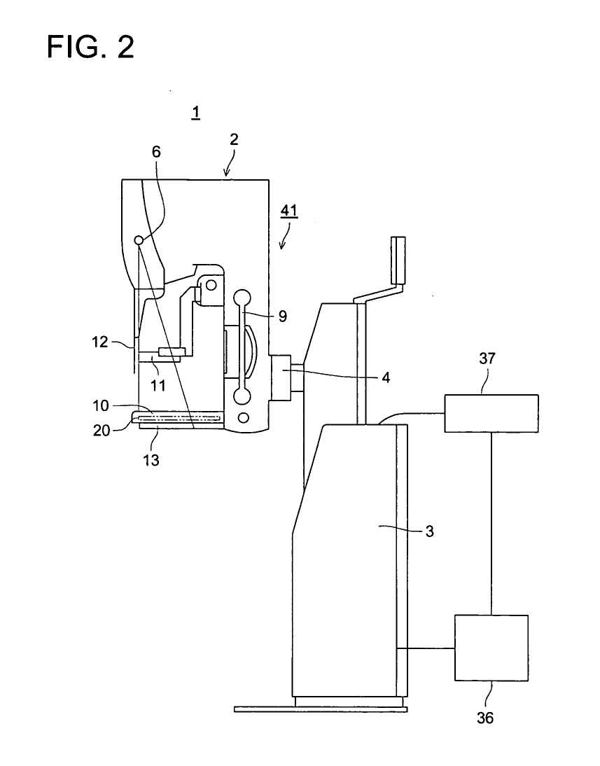

The embodiments of this invention are explained as follows by referring to drawings attached. FIG. 1 shows the side view of a radiographing apparatus and FIG. 2 shows a side view of a mammography unit of this invention respectively.

In the present embodiment, supporting member 5 equipped with phase contrast image support table 14 is attached detachably to radiographing unit 2 in mammography unit 41 of radiographing apparatus 1. The radiographing unit 2 is supported by backing shaft 4 equipped in backing base 3, and it is desirable that backing shaft 4 is attached to be moved up and down to adjust its height according to the requirements from variety of radiographing methods, differences of body form such as height of the examinee or a posture of examinee such as being on a wheelchair or not. The backing base 3 is connected with radioactive ray operation panel 37, which has a key for switching radiographing modes, and is connected to power supply 36 as a power source for the apparatus.

A radiation source 6, radiating a radioactive ray, is attached on upper side portion of a radiographing unit 2 and an x-ray tube whose focal size point is 30-2000 μm, widely used at general medical facilities, is used as a radiation source. In detail, an x-ray tube whose wavelength is about 0.1 nm is used. In this x-ray tube, a radioactive ray is radiated by energy conversion from kinetic energy to radiant energy, which is obtained by a collision of electrons generated by thermal excitation and accelerated by high voltage against a cathode. When a radiation image is taken, this accelerated voltage corresponding to an exposure time, volume of electrons generated corresponding to a tube current and radioactive ray radiated time corresponding to exposure time are set. Selecting a material of an anode from a group of copper, molybdenum, rhodium, and tungsten etc can change a radiation energy spectrum radiated. When copper, molybdenum, or rhodium is used as material of the anode of the x-ray tube, radioactive ray having a relatively low energy distribution line spectrum with narrow distribution bandwidth can be obtained and by utilizing this characteristics, it is used for mammography having functions such as radiation diffraction crystal analysis and reading a detailed structure. When tungsten is used for an anode of a x-ray tube, radioactive ray obtained has relatively wide spectrum with high energy and can be used for a radiation photograph for chest, abdominal and head portion of human body, and nondestructive inspection in general industrial application. It is distinctive that radiation dose irradiated is high when used in medical and industrial applications. In these cases, large number of electrons hit an anode terminal at high speed accordingly temperature of the anode terminal goes up high enough to melt anode plate itself. In order to avoid this drawback, an anode is rotated to change the collision spot. In other words, it is normal to use a rotary anode terminal. It is desirable to use molybdenum, rhodium or tungsten for rotary anode in x-ray tube since radiographing apparatus shown in this embodiment is designed for medical applications or nondestructive inspections.

Here, a focal point of radioactive ray is considered a window viewed from a photographic object to take out radioactive ray generated by collision of electrons against a rotary anode in an x-ray tube. In general, a shape of a focal point is square and length of a side of the square corresponds to the size of the focal point. When a shape of focal point is a circle, a size of the focal point corresponds to a diameter of the circle and when the shape of the focal point is rectangle, a size of the focal point corresponds to a length of the shorter side. Measuring methods such as a method by a pinhole camera and a method by a micro-test chart are explained in JIS (Japanese Industrial Standards) Z4704.

A lower limit of focal point size of radioactive ray used is determined by the condition that a certain level of radiation dose is required to obtain a clear image without blur, and an upper limit of focal point size of radioactive ray used is determined by following factors such as a distance between the photographic object 8 and radiation image information detection member 20, a distance between the focal point and photographic object 8 or physical characteristics of radioactive ray to obtain a highly sharp image by emphasizing, on an optimized condition, edges (phase contrast emphasized) on a boundary portion of photographic object 8 caused by the diffraction effect of radioactive ray. In order to practice a phase contrast image radiographing at normal medical facilities, the size of focal point is required to be in a range from 30 μm to 300 μm, and a range from 30 μm to 200 μm is more preferable.

Grip bar 9, which an examinee uses to hold its body may be attached on both sides of radiographing unit 2 and face guard 12 is attached on the side facing toward the examinee on radiographing unit 2. Photographic object table 10 which supports photographic object 8 from the lower side and pressure plate 11, which gives pressure to hold the photographic object 8 from upper side, are arranged at the position perpendicularly under radiation source 6 to move up and down freely. Further more, it is recommended that photographic object table 10 is a rectangular frame or a rectangular frame with a transparent and thin plastic plate pasted on it.

In this embodiment, supporting member 5 having thereon phase contrast image support table 14 is furnished detachably to the main body of mammography unit 41. Accordingly, it may be removed when it is not used.

When used, in this embodiment, one or a plural phase contrast image support tables 14 are attached on supporting member 5 to be located at a point on a line drawn about perpendicularly from radiation source 6, to face radiation source 6. On each phase contrast image support table 14, radioactive ray image information detection member 20 as a detection means to detect radioactive ray image information based on radioactive ray transmitted through a photographic object 8, is installed detachably. A radioactive ray radiated from a radiation source 6, passing through a photographic object 8, can be observed as radiation energy (radiation image information) on the radiation image information detection member 20, having necessary area to detect the radioactive ray.

Besides phase contrast image support tables, absorption contrast image support table 13 that supports the radiation image information detection member 20 is provided so that it may touch the bottom of photographic object support table 10.

And in this embodiment, for example, an absorption contrast image support table 13 is arranged on lower surface of photographic object support table 10 arranged at a position R1 which corresponds to 55-70 cm from radiation source 6, and a phase contrast image support table 14a is arranged at position R2 that is away from photographic object table 10 by a distance that is 0.5-1.5 times the distance of R1, and phase contrast image support table 14b is arranged at position R3 that is lower than and is away from phase contrast image support table 14a by a distance that is 0.3-1.0 times the distance R1.

In this embodiment, an absorption contrast image support table 13 is mounted on lower side surface of photographic object table 10 without space in between however, it is possible to install a radiation image information detection member 20 at any position, in the vicinity of photographing object table 10, for example on the upper surface of photographing object table 10 or inside of it, as far as the radiation image information detection member is located at the other side of radiation source of photographing object 8 and within the range which absorption contrast image can be photographed. Also it is not necessary to have a photographing object table 10 and an absorption contrast image support table 13 separately, since it is possible to make photographing object table 10 to function as an absorption contrast image support table 13 by making photographing object table 10 to support radiation image information detection member 20.

A grid (not shown) for intercepting scattering radioactive ray is provided on radiation source 6 side of radiation image information detection member 20 to prevent that However the grid may not be provided at radiation source 6 side, on the radiation image information detection member 20 attached to a phase contrast image support table 14 since scattering radiation dose is decreased as the distance from photographing object table 8 increases.

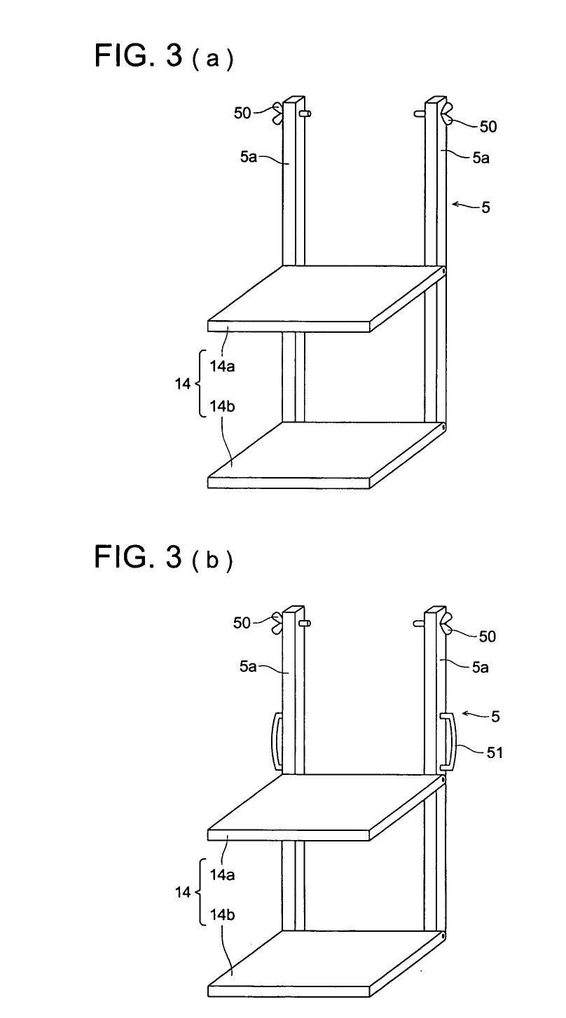

A supporting member 5, as shown in FIG. 3(a), is hold to a radiographing unit 2 as to fix the upper potion of supporting member 5a to lower portion of the radiographing unit 2 by putting a clamping component 50 to lower portion of the radiographing unit 2. As mentioned above, a supporting member 5 can be mounted or dismounted simply and surely by using clamping component 50. It is believed that a burden to attach or detach a supporting member 5 can be decreased, and moving and removing can be done safely since the weight of supporting member 5 is as light as 30 Kg or less.

And, a grip handle 51 is provided at the center portion of a supporting member 5 as shown in FIG. 3(b). A supporting member 5 can be simply and surely mounted or dismounted by using a grip handle 51 when conducting mounting or dismounting supporting member 5 for radiographing unit 2. So it is believed that a burden to mounted or dismounted a supporting member 5 can be decreased and it can be done safely since a supporting member 5 has a grip handle.

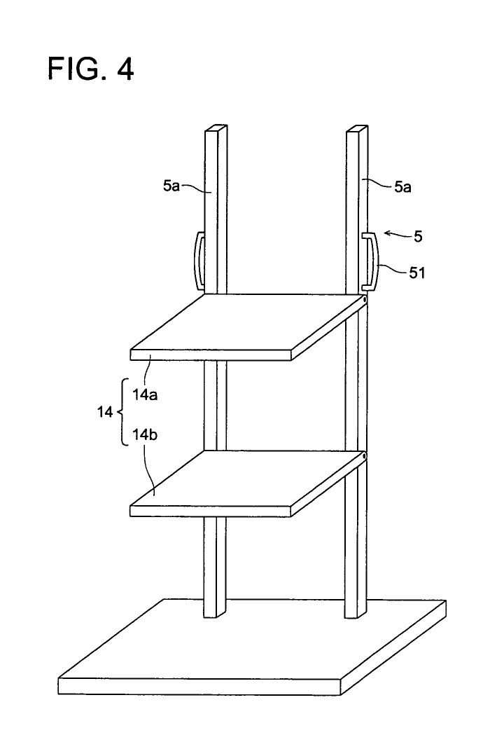

Incidentally, as another example, as shown in FIG. 4, supporting member 5 can be placed below the radiographing unit 2 without being detachably mounted on the radiographing unit 2. In this case, when the supporting member 5 is provided with casters, it may be convenient for an operator.

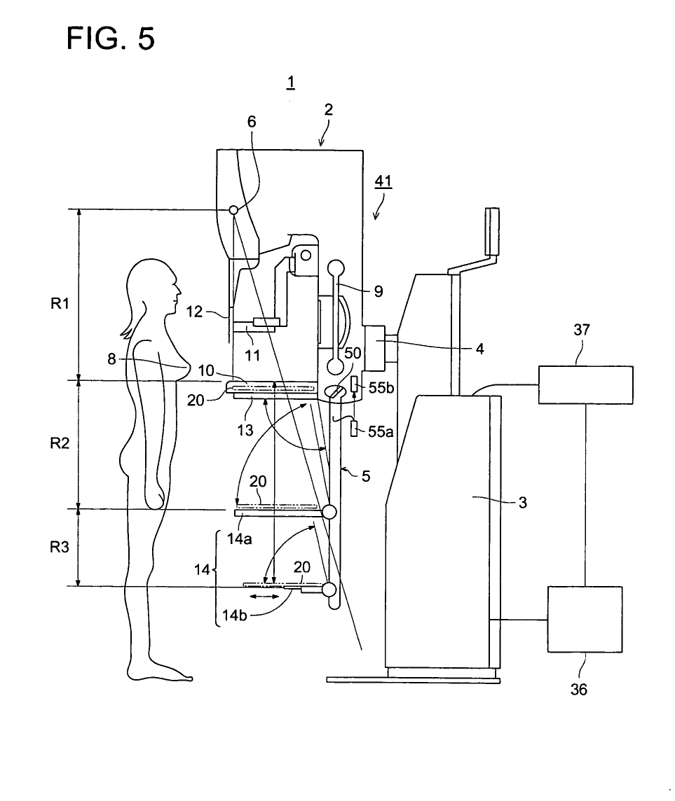

And supporting member 5 and main body of mammography unit 41 as shown in FIG. 5, are connected electrically. This electrical connection is done by attaching a supporting member 5 to radiographing unit 2 and then by connecting electric connector 55a on the supporting member 5 to electric connector 55b on the radiographing unit 2. As explained above, as supporting member 5 and main body of a mammography unit 41 are connected electrically, if electric connector 55a which detects a supporting member 5 attached or attached or detached and electric connector 55b having a sensor are provided, then the status of supporting member 5 attached or detached can be recognized on the part of main body of a mammography unit 41 and a malfunction can be prevented in advance.

Phase contrast image support table 14 attached on the supporting member 5 can be driven by remote control from radiographing unit 2 because the supporting member 5 and the radiographing unit 2 are connected electrically, and it is possible to move the phase contrast support table 14 to a certain position or retreat when the phase contrast image support table 14 is not used.

In this embodiment, the phase contrast image support table 14 attached on the supporting member 5 can be retreated from the point where a phase contrast image is photographed to secure the space on the opposite side of the radiation source against the photographic object table 10.

In regard to the system, which makes it possible to retreat, there are several systems such as a detachable system, a foldable system and a sliding system. In this embodiment, the foldable system is employed for phase contrast image support table 14 so that the phase contrast image support table 14 can be retreated from radiographing position without detaching it. In other words, the burden on the operator can be decreased since phase contrast image support table 14 can be retreated to the position, where it is not going to be an obstacle to the examinee without mounting or dismounting conducts. Also it is not necessary to secure the space to keep phase contrast image support table 14 since it is not necessary to dismount the phase contrast image support table 14.

When an absorption contrast image is radiographed, in other words, when the phase contrast image support table is not used, mounting and dismounting workload can be decreased since the phase contrast image table 14 can be retreated to the position where the phase contrast image support table is not going to be an obstacle to the examinee, without dismounting the supporting member 5.

In this embodiment, supporting member 5 having phase contrast image support table 14 can be attached to the mammography unit 41 as shown in FIG. 2. Specifications for the mammography unit 41 are as follows. However this invention is not limited to these specifications.

In regard to setting a range for radiographing condition, when a small focal point is selected, the ranges are 2 mAs-200 mAs and 22 kV-35 kV, when a large sized focal point is selected, they are 2 mAs-600 mAs and 22 kV-39 kV.

In regard to an additional filter, when tube voltage is set at 35 kV, it is 0.03 mmMo (first half value layer (minimum): 0.39 mmA1) or 0.02 mmRh (first half value layer (minimum): 0.47 mmA1), when tube voltage is set at 39 kV, it is 0.02 mmRh (first half value layer (minimum): 0.49 mmA1). Here, the first half value is measured by using Aluminum plate wherein the purity of Aluminum is 99.9%, or more.

Output rating of X-ray high voltage apparatus is 2.8 kW (30 kV, 92 mA, 6.0 second). The setting condition for X-ray exposure range on the X-ray high voltage apparatus is as follows. The setting range for kV is from 22 kV to 39 kV (1 kV step) and that for mAs is from 2 mAs to 600 mAs.

Automatic Exposure Control (AEC) function is available in every radiographing method such as contact radiographing, conventional macrophotograhing and Bucky's photographing. Ranges where automatic exposure control functions are as follows. The range is from 24 kV to 34 kV in kV setting and controllable mAs range is from 2 mAs to 600 mAs for large sized focal point and from 2 mAs to 200 mAs for small sized focal point, and the number of steps of film density to be set is 15 steps.

In X-ray exposure control, an automatic kV/mAs control, kV pre-setting/automatic mAs control and kV/mAs pre-setting are available.

The X-ray exposure interlock system works when any abnormal situation is detected, in the control circuit for the rotary anode in X-ray tube, the control for a filament in s-ray tube, the power supply of main inverter and in the high voltage circuit, when Mo/Rh filter is not located at specified position, when Automatic Exposure Control (AEC) is selected, and set to be out of range for possible radiography condition, when a temperature sensor of X-ray tube case works, when X-ray focal point size does not agree with irradiation field limit plate or when an optical irradiation field mirror is not retreated.

With respect to a photographing table, in terms of C shaped arm movements, its up and down stroke is about 69 cm, a rotation angle is about 180°-−(minus) 150° (electrically-powered). A lock system is electro-magnetic lock system, off-lock type and a display for rotating angle starts from 180° C. to −(minus) 150° C., 1° step. The rotation angle can be set in manual mode by using a position setting switches located on the side plate of C shaped arm, and also it is set by using switch CC, ML and MLO on the photographic stand automatically in automatic mode.

X-ray tube apparatus model name is DRX-B3856HD-Mo, focal point size is 0.1 mm in small size focal point, 0.3 mm in large size focal point, an anode plate material is Mo (molybdenum), a target angle is 10° in small size focal point, 10° in large size focal point, number of rotation of anode plate is 9700 rpm, maximum thermal capacity for an anode plate is 300 kHU (210 kJ), X-ray radiation outlet is made of Be (beryllium), and intrinsic filter is about 1.0 mmBe.

With respect to connection cables, its effective length between radiographing unit and X-ray unit (control unit) is about 9 m, that between X-ray high voltage unit (X-ray operation panel) and X-ray high voltage unit (control unit) is about 9 m that between power switchboard and X-ray high voltage unit (control unit) is about 9.

In power source conditions, the power source is singe-phase alternating current power, nominal power voltage corresponds to 200 V, 208 V, 220 V, 230 V and 240 V, the frequency of power supply is 50 Hz or 60 Hz, a maximum permissible range of voltage fluctuation is 10% of nominal voltage when no-loading, a permissible power line impedance is not more than 0.33 ohms when 200 V, not more than 0.34 ohms when 208 V, not more than 0.36 ohms when 220 V, 0.38 ohms when 230 V and not more than 0.4 ohms when 240 V. And recommended capacity for power supply is about 4.1 kVA. Maximum alternating current power supply is 20.5 A when voltage is 200V−(minus) 10% (180V).

Grounding (earth) condition should conform to D-class grounding (earth) construction in (The technical standards of electrical equipment by Ministerial ordinance)

Required ceiling height is 230 cm.

In regard to environmental conditions, ambient temperature is from 10° to 40° C., relative humidity is from 30 to 85% (non-condensation) and air pressure is from 70 to 106 kPa, as working environmental conditions, and as storage environmental conditions, ambient temperature is from −(minus) 10 to 60° C., relative humidity is from 10 to 90% (non-condensation) and air pressure is from 50 to 106 kPa.

In regard to the conditions for use and storage of the apparatus, following items from item (a) to item (m) should be avoided for the use and storage: (a) out of environmental condition specified above, (b) place filled with harmful gas (c) high humidity place such as place filled with steam, (d) a place where water drops drip on the apparatus, (e) dusty place including a cloud of dust, (f) place with much oily steam, (g) place where the apparatus is exposed to salty air, (h) place with explosive gas, (i) place where excessive vibration or impact is expected, (j) a place where an inclination of more than 0.18 radian (10°) is present, (k) place where high fluctuation of power voltage is expected, (1) place where excessive power voltage drop occurs when non-loading, (m) place where direct sun light is expected.

Following is the classification of the apparatus. (1) Classification for protection form for electric shock corresponds to apparatus class I, (2) Classification for protection level for electric shock is B-type mounting section, (3) Classification of level for harmful water invasion corresponds to normal equipment, (4) Classification of safety use in the environment of air, inflammable anesthesia gas or oxygen nitrous sub oxide corresponds to product not suitable for use in the environment of air, inflammable anesthesia gas or oxygen/nitrous sub oxide. (5) Classification of operation mode is continuous operation with intermission load.

In regard to leak current, under the condition that temperature is 10-40° C., relative humidity is 30-85% and air pressure is 70-106 kPa, earth leak current is not more than 5 mA, armor leak current is not more than 0.1 mA, an examinee leak current is no more than 0.1 mA, when power source is normal condition. In the case of single failure condition, earth leak current is not more than 10 mA, armor leak current is not more than 0.5 mA and an examinee leak current is not more than 0.5 mA.

In regard to regulations, the apparatus above conforms following regulations.

(1) JIS (Japanese Industrial Standard) Z-4701-1997 “Medical X-ray apparatus general roles”

(2) Ministry of Health and Welfare, Medical Machine No. 149

(3) IEC 60601-1 (1988)/A1 (1995) (General rules for safety of medical electric machine)

(4) IEC-60601-1-1 (1992)/A1 “Requirements for safety of medical electric systems”

(5) IEC 60601-1-2 (1993) “Electro magnetic conformity (EMC)”

(6) IEC 60601-1-3 (1994) “Protection for radiation of diagnostic X-ray apparatus”

(7) IEC60601-1-4 (1996) “Programmable electric medical systems” (8) IEC60601-2-7 (1987) (Ed2 (1998) “conformity” “Safety of X-ray high voltage apparatus”

(9) IEC-60601-2-45 (1998) “X-ray (radiation) source”

(10) IEC-60601-2-32 (1994) “Individual regulations for safety of X-ray relevant apparatus”

(11) IEC-60601-2-45 (1998) “Safety of mammography unit”

Apparatus conforming to regulations described above is used Preferably.

A radiation image information detection member used in this embodiment, it is possible to use the following components: a combination of x-ray intensifier foil and silver halide photographic film; a florescent screen for photo-stimulated luminescence; a radiation image information reader wherein a scintillator to convert radiation energy to light and optical semiconductor element to read its light are arrayed two-dimensionally; a radiation image information reader in which is a photoconductor to convert radiation energy to electric signals directly and a semiconductor device to read the electric signals are arrayed two-dimensionally; a radiation image information reader in which a set or a plurality of sets each including sintillator to convert radioactive ray to light and a lens to converge light on CCD and CMOS, are arrayed; or a sintillator to convert radioactive ray to lights and a radiation image information reader which leads the light to CCD or CMOS by an optical fiber to convert into electric signals.

In the case that the radiation image information detection member is as mentioned above, a radiation dose detector (for example, photo-timer) as a means to detect the radiation dose, may also be attached on the backside of the radiation image information detection member. In the case that the radiation energy can be taken out directly from radiation image information detection member such as flat panel detector, it may be allowed to have the same function as that of the radiation dose detector mentioned above on the radiation image information detection member itself without a radiation dose detector.

In this embodiment, a combination of X-ray intensifier foil and a silver halide photographic film representing a radiation image information detection member is called SF-system (screen film system). X-ray intensifier foil includes phosphor using rare earth element such as calcium tungsten and gadolinium oxide sulfide, and converts radiation energy to light emitted in blue or green. Especially, as for an intensifying screen using a phosphor using rare earth element, the technologies disclosed in TOKKAKIHEI No. 6-67365 may be used. A silver halide photographic film having photosensitive emulsion applied on one side or both side of the film support is preferable to be used. Especially in the case of a double emulsion film, it is desirable to use photosensitive material wherein photographic characteristic of an emulsion layer on one side is different from that of an emulsion layer on the other side both sandwiching the film support. Also it is desirable to use a photographic film having an absorption layer to absorb crossover light in between both sides of the emulsion layers of a double emulsion film. This embodiment, films in all size from 8×10 inch to 20×24 inch can be used as single sided and double sided film used in this embodiment. These silver halide photosensitive material is outlined in TOKKAIHEI No. 6-67365 and, for example, Principal of Photographic Science and Engineering (Corona Publishing Co., Ltd, authored by The Society of Photographic Science and Technologies of Japan). In regard to photographic process, it is possible to improve an average gradation by raising the temperature of photographic process or extending the process time, however, principally, it is desirable to follow the photographic process specified by the film manufacturer.

What is called photo-stimulated luminescence type fluorescent screen is the screen from which visible ray emission corresponding to the X-ray intensity irradiated is induced by irradiating visible ray or infrared ray to the screen after the X-ray irradiation. In other words, the photo-stimulated luminescence type fluorescent screen is placed as a radiation image information detection member and then the fluorescent screen is moved to laser reader apparatus to read simulated emission after irradiating X-ray to the fluorescent screen then the emitted light that is read is converted to electric signal by a photo multiplier, which results in acquisition of the electric signals of radiation image. After the electric signals (image data) are processed properly, they are displayed on image display means such as a monitor, etc, or hard copies of radiation imges or obtained by using an image output means such as a laser imager. When macro-photographing, the image can be displayed automatically in about real size on monitor display or hard copied by specifying the magnifying power in advance. Regarding a radiation image information detection member using photo-stimulated luminescence type fluorescent screen, the fluorescent screen and image visualization technologies, such as photo-stimulated luminescence read-out, en-visible technologies, disclosed in TOKUKAI 2000-245721, can be used in this embodiment.

In regard to the radiation image information detection members converting radiation to electric signals explained above, technologies disclosed in TOKUGANHEI No. 11-49080 or Handbook of Medical Imaging Volume 1, Chapter 4 Flat panel imager for digital radiography (ed. R. V. Matter etc., SPIE Press, Bellingham, 2000) can be used in this embodiment. In these cases, radiation image information detection member can play a role of a detector for radiation dose and electric signals of radiation image obtained by the radiation image information detection member can be processed properly and displayed on the monitor or hard-copied for medical image diagnosis.

Furthermore, in the case of macro photographing, in “a phase image radiographing mode” for obtaining a phase contrast image, it is possible to display by changing the image to life-size when outputting the obtained a radiation image on the monitor or on a hard copy such as a photographic film.

In regard to methods of a hardcopy, those obtained by using silver halide photographic sensitive material and by processing with an automatic processor, those processed by heating after exposure on silver halide photographic sensitive material by laser ray corresponding to the radiation image information, and those on which images are formed by heating corresponding to radiation image information, are preferable embodiments. Also a solid inkjet record method which jets liquid state material from the nozzle to draw the image at room temperature, a inkjet recording method which jets liquid such as dye or color at room temperature from a nozzle to draw the image, a method to draw the image by fixing the ink sublimated on the ink ribbon by heating to draw the image and an ablation image forming method to form an image by evaporating a sheet coated entirely with carbon by irradiating laser beam based on the image information, are used to make hard copies, which can be used.

Also, a control unit for controlling each part is equipped on the radiation image photographic apparatus 1 in this embodiment. Radiation source 6, pressure board 11, absorption contrast image support table 13, a driving device of phase contrast support table 14 and radiation operation panel 37 as an input device to specify the photographic mode are connected to this control unit. A key to specify a mode selectively is provided on the radiation operation panel 37, which includes “normal photographic mode” which is done by attaching a radiation image information detection member 20 to an absorption contrast image support table 13, “first phase image photographic mode” which is done by attaching a radiation image information detection member 20 to phase contrast photographic support table 14a, “second phase image photographic mode” which is done by attaching a radiation image information detection member 20 to the phase contrast photographic support table 14b, “normal photographic mode” done by a absorption contrast image support table 13 and a plural of “phase image photographic mode” which corresponds to different kinds of magnification. Further, as an input device, a keyboard, a magnetic card reader, a bar code reader a HIS (Hospital Information Systems) etc may be utilized, and which may be provided separately from the radiation operation panel 37.

Also, an image output unit to output photographic image by a printer etc and/or an image display unit to display the image on a display screen are connected to the operation unit.

As mentioned above, according to the invention described in Item 1, as a supporting member with a phase contrast image support table attached, can be mounted on and dismounted from the main body of mammography unit freely, when radiographing an absorption contrast image, in other words, when a phase contrast image support table is not used, it is prevented that the phase contrast image support table becomes an obstacle to the examinee, accordingly the burden to the examinee can be decreased.

According to the invention described in Item 2, as the supporting member is so light as to be 30 Kg or less, the workload for installation and removal can be decreased and operation thereof can be done safely.

According to the invention described in Item 3, having a grip handle on the supporting member can make the workload decreased and operation thereof can be done safely.

According to the invention of Item 4, the supporting member and the mammography unit are connected electrically so that a status of the supporting member, attached or detached, can be detected at the mammography unit in the case that a sensor to detect the supporting member attached on or detached from the mammography unit is provided, and accordingly, a malfunction can be prevented. Furthermore, the phase contrast image support table attached on the supporting member can be driven from the mammography unit by remote control operation, and thereby, the phase contrast image support table can be moved to a certain position or it can be retreated when it is not used.

According to the invention of item 5, when an absorption contrast image is radiographed, in other words, when the phase contrast support image table is not used, it can be retreated to the space to prevent to become an ngly it reduces a load ase contrast support table

What is claimed is:

1. A radiographing apparatus for mammography, comprising:
a radiographing main body including:
a radiation source to emit radiation;
a breast table on which a beast to be radiographed is placed, the breast table located opposite to the radiation source, and
a first detector supporting rack to support a detector to receive radiation having passed through the breast when the beast is radiographed as an absorption contrast image,
a supporting stand to support the radiographing main body at an upper position so as to provide a space at a lower position; and
a detector supporting member shiftably provided in the space at the lower position of the supporting stand and having a second detector supporting rack to support a detector when the beast is radiographed as a phase contrast image.

2. The radiographing apparatus of claim 1, wherein the supporting stand supports the radiographing main body in such a way that the radiographing main body is movable upwardly or downwardly.

3. The radiographing apparatus of claim 1, wherein the detector supporting member is detachably attached to the radiographing main body.

4. The radiographing apparatus of claim 1, wherein the detector supporting member is shiftably placed at the lower position of the supporting stand.

5. The radiographing apparatus of claim 1, wherein the second is hinged so as to rotate between a horizontal attitude and a vertical attitude.

6. The radiographing apparatus of claim 1, wherein the detector supporting member further has a third detector supporting rack provided below the second supporting rack.

7. The radiographing apparatus of claim 1, wherein the detector supporting member is provided with a handle.

8. The radiographing apparatus of claim 1, wherein the detector supporting member has a weight of 30 Kg or less.

9. The radiographing apparatus of claim 1, wherein the detector supporting member is electrically connected with the radiographing main body.
